(19) United States
(12) Patent Application Publication
(10) Pub. No.: US 2026/0047811 A1
(43) Pub. Date: Feb. 19, 2026

(54) HEAD FIXATION DEVICE

(71) Applicant: FUJIFILM Corporation, Tokyo (JP)

(72) Inventors: Hiroki SADAMARU, Tokyo (JP); Yuta INOUE, Tokyo (JP)

(73) Assignee: FUJIFILM Corporation, Tokyo (JP)

(21) Appl. No.: 19/290,411

(22) Filed: Aug. 5, 2025

(30) Foreign Application Priority Data
Aug. 13, 2024 (JP) 2024-135092

(51) Int. Cl. A61B 6/04

(52) U.S. Cl. CPC A61B 6/0407

(57) ABSTRACT

Provided is a head fixation device that can accurately register a head and has a simple configuration. A head fixation device for use in a medical imaging apparatus, including: an occipital region placement portion on which an occipital region of a head of a subject is placed; and a temporal region support portion that is continuously provided from both sides of the occipital region placement portion and supports both sides of a temporal region of the head, in which the temporal region support portion includes a viewing window portion which is configured so that a region of the temporal region, which is a reference in a case of capturing a tomographic image of the head, is visually recognizable in a state where the occipital region is placed on the occipital region placement portion.

### CROSS-REFERENCE TO RELATED APPLICATION

The present application claims priority under 35 U.S.C § 119(a) to Japanese Patent Application No. 2024-135092 filed on Aug. 13, 2024, which is hereby expressly incorporated by reference, in its entirety, into the present application.

### BACKGROUND OF THE INVENTION

### 1. Field of the Invention

The present invention relates to a head fixation device for use in a medical imaging apparatus.

### 2. Description of the Related Art

In a medical imaging apparatus, a tomographic image of a subject (patient) is acquired by irradiating the subject placed on a bed with electromagnetic waves (RF pulses) or X-rays. In order to obtain an appropriate tomographic image, it is necessary to place the subject at an appropriate position on the bed.

For example, in a case of capturing a tomographic image of a head of the subject, it is necessary to fix the head at a position in accordance with a predetermined reference line in order to obtain a desired tomographic image. For this reason, a head fixation device (also referred to as a head receiver, a headrest, a head holder, or a head cradle) for fixing the head may be used.

For example, JP3808215B discloses a head fixation tool comprising an air mat on which a head of a patient is placed, the air mat being capable of contracting a side portion that supports a temporal region, a flexible forehead fixation unit that supports a forehead of the patient, a flexible chin fixation unit that supports a chin of the patient, and a plurality of pads provided on a bottom portion of the air mat, a forehead abutting portion of the forehead fixation unit, and a chin abutting portion of the chin fixation unit, respectively.

In addition, for example, JP1997-075333A (JPH09-075333A) discloses a headrest comprising a substantially annular head receiving plate in which a first rotation shaft is formed at a lower end portion, a second rotation shaft formed at one end portion of a bed top plate, and a link member of which one end portion is pivotally attached to the first rotation shaft so as to be movable rotationally and the other end portion is pivotally attached to the second rotation shaft so as to be movable rotationally. The head can be fixed in accordance with a predetermined reference line by moving the link member rotationally about the first rotation shaft and the second rotation shaft in a state where the occipital region is dropped into an annular hole of the head receiving plate.

Further, for example, JP2021-168912A discloses a head holder comprising a head cradle, an inclination adjustment mechanism having a plurality of lock positions disposed below a first end portion of the head cradle, a table mount extending from a second end portion of the head cradle, and an inclination adjustment bar extending from the table mount through the inclination adjustment mechanism. The head cradle is fixed to a table on which the subject is placed via a table mount, and the head is placed on the head cradle. By adjusting the angle of the inclination adjustment bar by the inclination adjustment mechanism, the inclination angle of the head holder can be adjusted.

### SUMMARY OF THE INVENTION

However, the head fixation tool disclosed in JP3808215B has a problem in that the registration of the head with the reference line is not considered. In addition, the headrest disclosed in JP1997-075333A (JPH09-075333A) and the head holder disclosed in JP2021-168912A consider the registration of the head, but have a problem of a complicated structure.

The present invention has been made in order to solve the above-described problems, and an object of the present invention is to provide a head fixation device that can accurately register the head and has a simple configuration.

A head fixation device according to a first aspect is a head fixation device for use in a medical imaging apparatus, comprising: an occipital region placement portion on which an occipital region of a head of a subject is placed; and a temporal region support portion that is continuously provided from both sides of the occipital region placement portion and supports both sides of a temporal region of the head, in which the temporal region support portion includes a viewing window portion which is configured so that a region of the temporal region, which is a reference in a case of capturing a tomographic image of the head, is visually recognizable in a state where the occipital region is placed on the occipital region placement portion.

According to a second aspect, in the head fixation device according to the first aspect, the region of the temporal region that is visually recognizable is a region from an orbital cavity to an external auditory canal.

According to a third aspect, in the head fixation device according to the first or second aspect, the reference is any one of an orbitomeatal base line, a Reid's base line, an anterior commissure-posterior commissure line, a superior orbitomeatal line, or an auricular line.

According to a fourth aspect, in the head fixation device according to any one of the first to third aspects, the viewing window portion is an opening or a cutout provided in the temporal region support portion.

According to a fifth aspect, in the head fixation device according to the fourth aspect, the opening or the cutout is formed in a curve.

According to a sixth aspect, in the head fixation device according to any one of the first to fifth aspects, the temporal region support portion has breathability.

According to a seventh aspect, in the head fixation device according to the sixth aspect, the breathability of the temporal region support portion is achieved by a plurality of ventilation holes.

According to an eighth aspect, in the head fixation device according to any one of the first to third aspects, the viewing window portion has a net shape provided on the temporal region support portion.

According to a ninth aspect, the head fixation device according to any one of the first to eighth aspects further comprises: a lens protection member that protects eyes of the subject.

According to a tenth aspect, in the head fixation device according to the ninth aspect, the lens protection member is attachably and detachably disposed on the temporal region support portion.

According to an eleventh aspect, the head fixation device according to any one of the first to tenth aspects further comprises: an upper arm placement portion on which an upper arm portion of the subject is placed.

According to a twelfth aspect, in the head fixation device according to the eleventh aspect, the upper arm placement portion is a separate body from the occipital region placement portion and the temporal region support portion.

According to a thirteenth aspect, in the head fixation device according to the twelfth aspect, in a case where the head fixation device is used, at least a part of the upper arm placement portion is disposed below the occipital region placement portion.

According to a fourteenth aspect, in the head fixation device according to the thirteenth aspect, in a case where the head fixation device is used, among a plurality of end portions of the upper arm placement portion, an end portion located below the occipital region placement portion is curved.

According to a fifteenth aspect, in the head fixation device according to the fourteenth aspect, the curved end portion of the upper arm placement portion located below the occipital region placement portion extends over three or more tomographic images.

Since it is possible to suppress the operation sound in a case where the operator and the subject converse with each other during imaging by the medical imaging apparatus, it is possible for the operator and the subject to smoothly converse with each other.

### BRIEF DESCRIPTION OF THE DRAWINGS

FIG. 1 is an outline configuration diagram of a medical imaging apparatus.

FIG. 2 is a perspective view of a head fixation device according to a first embodiment.

FIG. 3 is a plan view and a front view of the head fixation device according to the first embodiment.

FIG. 4 is a diagram for describing a viewing window portion.

FIG. 5 is a diagram showing a modification example of the head fixation device.

FIG. 6 is a diagram showing a modification example of the head fixation device.

FIG. 7 is a diagram showing an example of the head fixation device including a lens protection member.

FIG. 8 is a diagram showing an example of the head fixation device including an angle marker.

FIG. 9 is an exploded perspective view of a combination of the head fixation device and an arm fixation device according to a second embodiment.

FIG. 10 is an assembly perspective view of the combination of the head fixation device and the arm fixation device according to the second embodiment.

FIG. 11 is a front view of the combination of the head fixation device and the arm fixation device according to the second embodiment.

FIG. 12 is a plan view of the arm fixation device.

FIG. 13 is a perspective view of a combination of a head fixation device and an arm fixation device according to the related art.

FIG. 14 is a diagram showing an example of a head tomographic image captured using the head fixation device and the arm fixation device according to the related art.

FIG. 15 is a diagram showing an example of a head tomographic image captured using the head fixation device and the arm fixation device according to the second embodiment.

FIG. 16 is a perspective view of the arm fixation device.

### DESCRIPTION OF THE PREFERRED EMBODIMENTS

### Outline Configuration of Medical Imaging Apparatus

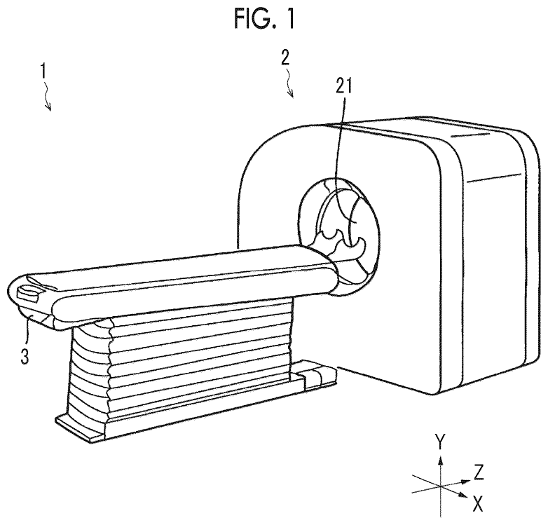

First, a general outline configuration of a medical device to which a head fixation device according to the present disclosure can be applied will be described with reference to FIG. 1. The head fixation device according to the present disclosure can be applied to the medical device such as a medical imaging apparatus that requires accurate registration of the head. Examples of the medical imaging apparatus include an X-ray computed tomography (CT) apparatus, a nuclear magnetic resonance imaging (MRI) apparatus, and the like. Hereinafter, a case where the medical imaging apparatus is an X-ray CT apparatus will be described, but the medical imaging apparatus is not limited to this. In the accompanying drawings, the three-dimensional coordinate system shows an example of a definition of a direction in the medical imaging apparatus. An X axis, a Y axis, and a Z axis of the three-dimensional coordinate system are merely examples and the present invention is not limited thereto.

FIG. 1 is an external view of a medical imaging apparatus 1. As shown in FIG. 1, the medical imaging apparatus 1 comprises a gantry 2, a bed 3, and an operation unit (not shown). The gantry 2 incorporates a device that emits X-rays or radio frequency (RF) pulses, and is configured to be rotatable around the bed 3. In addition, an opening 21 through which the bed 3 can pass is formed near the center of the gantry 2. The bed 3 is formed in a long rectangular shape in a Z-axis direction. That is, the Z-axis direction is a longitudinal direction of the bed 3. A subject S is placed on the bed 3.

In the following description, in a state where the subject S is placed on the bed 3, the Z-axis direction is a body axis direction of the subject S, a Y-axis direction is a front-rear direction of the subject S (a direction parallel to a gravity direction), and an X-axis direction is a left-right direction of the subject S (a direction orthogonal to the gravity direction). In the following description, it is assumed that the medical imaging apparatus 1 captures tomographic images in a plurality of imaging cross sections while changing a position in the Z-axis direction (while changing a position in the body axis direction).

A head fixation device 100 is attachably and detachably attached to the bed 3. In a case of imaging in a head-first manner, for example, the head fixation device 100 is attached to an end portion of the bed 3 on a gantry 2 side in the longitudinal direction (Z-axis direction). In a case where the head is imaged, the subject S is placed on the bed 3 in a state where the head of the subject S is placed on the head fixation device 100. The operation unit is a device for an operator (not shown) to operate the medical imaging apparatus 1.

### First Embodiment

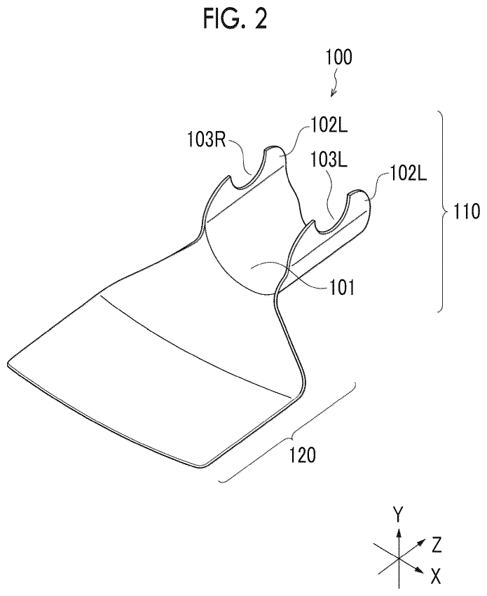

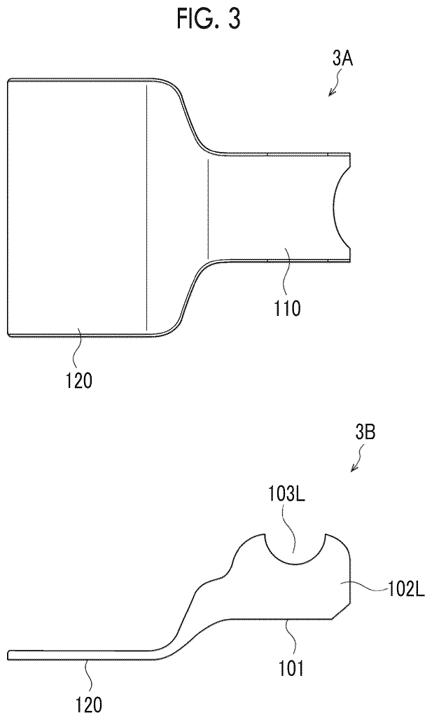

Next, a configuration of the head fixation device 100 according to the first embodiment will be described with reference to FIGS. 2 and 3. FIG. 2 is a perspective view of the head fixation device according to the first embodiment, a reference numeral 3A in FIG. 3 is a plan view of the head fixation device 100, and a reference numeral 3B in FIG. 3 is a front view of the head fixation device 100.

As shown in FIGS. 2 and 3, the head fixation device 100 comprises a head placement portion 110 and a bed connection portion 120. The head of the subject S is placed on the head placement portion 110. The bed connection portion 120 is continuously provided on the head placement portion 110, and the head fixation device 100 is attachably and detachably attached to the end portion (an end portion on a positive side in the Z-axis direction) of the bed 3 on the gantry 2 side in the longitudinal direction via the bed connection portion 120 (see FIG. 1). The shape of the bed connection portion 120 is appropriately changed according to the specifications of the bed 3.

A material of the head fixation device 100 is appropriately selected so as to suppress the influence on the image quality of the tomographic image. As the material of the head fixation device 100, a non-metal having a relatively low absorbance and refractive index with respect to the X-rays or the RF pulses emitted from the medical imaging apparatus 1 is preferable. Examples of the material of the head fixation device 100 include carbon (carbon fiber).

As shown in FIGS. 2 and 3, the head placement portion 110 has a shape similar to a partial circular tube (semicircular tube) as a whole. The head placement portion 110 comprises an occipital region placement portion 101, a pair of temporal region support portions 102, and a pair of viewing window portions 103. An occipital region of the subject S is placed on the occipital region placement portion 101. Preferably, the occipital region placement portion 101 has a curved surface along the curve of the occipital region of the subject S.

The pair of temporal region support portions 102 support both temporal regions of the head of the subject S. More specifically, the pair of temporal region support portions 102 include a right temporal region support portion 102R and a left temporal region support portion 102L. The right temporal region support portion 102R is continuously provided at one end portion of the occipital region placement portion 101 and supports a right temporal region of the head of the subject S. The left temporal region support portion 102L is continuously provided at the other end portion of the occipital region placement portion 101 on the side opposite to the one end portion at which the right temporal region support portion 102R is continuously provided. The left temporal region support portion 102L supports a left temporal region of the head of the subject S. Preferably, the right temporal region support portion 102R and the left temporal region support portion 102L have curved surfaces along the curve of the temporal region of the subject S.

The pair of viewing window portions 103 are provided on the pair of temporal region support portions 102. More specifically, the pair of viewing window portions 103 includes a right viewing window portion 103R provided on the right temporal region support portion 102R and a left viewing window portion 103L provided on the left temporal region support portion 102L. In a state where the occipital region is placed on the occipital region placement portion 101, the head fixation device 100 is configured such that the operator of the medical imaging apparatus 1 can visually recognize a region of the temporal region as a reference in a case of capturing a tomographic image of the head through the viewing window portion 103.

Preferably, the viewing window portion 103 formed in the temporal region support portion 102 is a cutout, an opening, or a mesh region or a punched region having a roughness to the extent that the operator can see through. In a case where the viewing window portion 103 is a cutout or an opening, preferably, the contour of the cutout or the opening is formed in a curve. This is to suppress the occurrence of the artifact in the tomographic image due to the linear contour line.

In FIGS. 2 and 3, for example, the temporal region support portion 102 has a U-shaped cutout as the viewing window portion 103. Specifically, the viewing window portion 103 shown in FIGS. 2 and 3 is formed by cutting out about one third of the height of the temporal region support portion 102 in the Y-axis direction from the end portion of the temporal region support portion 102 on the positive side in the Y-axis direction (up-down direction) to the negative side in the Y-axis direction and about half of the size in the Z-axis direction near the center in the Z-axis direction.

Hereinafter, the viewing window portion 103 will be described in more detail. First, in a case of capturing a tomographic image of the head (in a case of imaging in a head-first manner), the head is registered with a predetermined reference line, and the tomographic image is captured along the reference line. There are several types of reference lines, and the reference line is appropriately selected according to the type of the medical imaging apparatus 1 to be used, the part to be imaged in the head, and other conditions. A plane connecting the reference lines on both the left and right sides is the reference plane.

Examples of the reference line include an orbitomeatal base line, a Reid's base line, an anterior commissure-posterior commissure line, a superior orbitomeatal line, and an auricular line. The orbitomeatal base line (OML) is a line connecting the center of the orbital cavity and the center of the external auditory canal, and is also a line connecting the external canthus (the outer corner of the eye) and the center of the external auditory canal. The Reid's base line (RBL) is a line connecting the infraorbital margin and the upper margin of the external auditory canal. The anterior commissure-posterior commissure line (AC-PC) is a line connecting the anterior commissure and the posterior commissure. The superior orbitomeatal line (SML) is a line connecting the supraorbital margin and the center of the external auditory canal. The auricular line (ARL) is a line orthogonal to the RBL and passing through the center of the external auditory canal.

In a case of performing the registration, the bed 3 is moved toward the gantry 2 in a state where the head of the subject S is placed on the head fixation device 100 in a supine position. Subsequently, the subject S on the bed 3 is irradiated with a plurality of registration lasers from the gantry 2. In general, three lasers of a laser indicating an imaging cross section (slice plane), a laser indicating a center line of the bed 3 in the X-axis direction, and a laser indicating a height direction (Y-axis direction in FIG. 1) of the gantry 2 are irradiated as the registration lasers.

The operator operates the inclination angle of the gantry 2 with respect to the bed 3 while watching the registration lasers to register the head with a predetermined reference line. A large number of reference lines are determined based on the vicinity of a region from the orbital cavity (external canthus) to the external auditory canal in the temporal region.

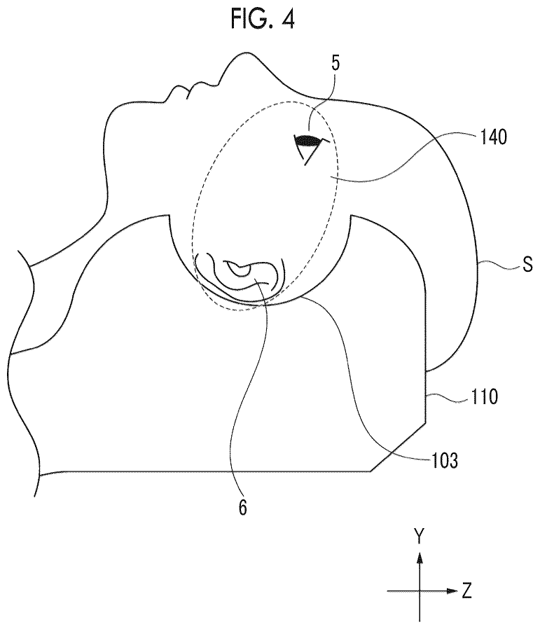

FIG. 4 shows a state where the head of the subject S is placed on the head fixation device 100 shown in FIGS. 2 and 3 in a supine position. As shown in FIG. 4, in a state where the head is placed on the head fixation device 100, the operator can visually recognize a region 140 from the orbital cavity to the external auditory canal of the temporal region of the subject S through the viewing window portion 103.

Therefore, during the registration, the operator can accurately register the head at a predetermined position while visually recognizing the region 140 from the orbital cavity to the external auditory canal of the temporal region through the viewing window portion 103. In addition, since this effect is realized by the viewing window portion 103 provided in the temporal region support portion 102, the structure of the head fixation device 100 is simple.

### Modification Example 1 of First Embodiment

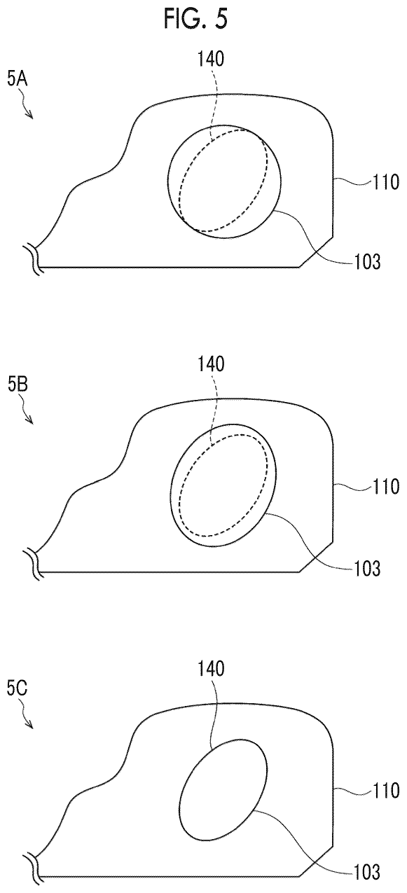

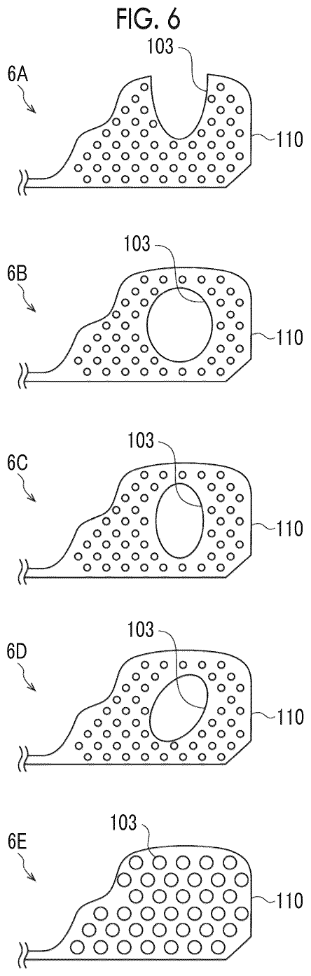

FIGS. 2 to 4 show a U-shaped cutout formed in the temporal region support portion 102 as an example of the viewing window portion 103. However, as long as the region 140 from the orbital cavity to the external auditory canal of the temporal region can be visually recognized through the viewing window portion 103, the shape and configuration of the viewing window portion 103 are not particularly limited. FIGS. 5 and 6 show further examples of the viewing window portion 103.

Reference numerals 5A to 5C in FIG. 5 show the head fixation device 100 (head placement portion 110) having an opening formed by hollowing out the temporal region support portion 102 as another example of the viewing window portion 103. In the example shown by the reference numeral 5A in FIG. 5, the head fixation device 100 has a circular opening as the viewing window portion 103. In the example shown by the reference numeral 5B in FIG. 5, the head fixation device 100 has an elliptical opening as the viewing window portion 103.

In the example shown by the reference numeral 5C in FIG. 5, similarly to the reference numeral 5B, the head fixation device 100 has an elliptical opening as the viewing window portion 103. As shown in FIG. 4, the region 140 has a shape close to an ellipse having a major axis slightly inclined toward the top of the head (the positive side of the Z-axis direction) with respect to a direction (Y-axis direction) perpendicular to the surface of the bed 3, in accordance with the positional relationship between the eye 5 and the ear 6. In the elliptical opening shown by reference numeral 5C, the major axis of the ellipse is slightly inclined toward the top of the head in accordance with the positional relationship between the eye 5 and the ear 6.

The size of the circular or elliptical opening is formed to be larger than the region 140 so that the region 140 from the orbital cavity to the external auditory canal of the temporal region of the subject S is exposed from the temporal region support portion 102. Therefore, even in these examples, in a case of the registration, the operator can accurately register the head at the predetermined position while visually recognizing the region 140 from the orbital cavity to the external auditory canal of the temporal region through the viewing window portion 103.

Reference numerals 6A to 6E in FIG. 6 show further examples of the head fixation device 100. In the example shown by the reference numeral 6A in FIG. 6, in the head fixation device 100 shown in FIGS. 2 to 4, a large number of through-holes are formed in the temporal region support portion 102. In the examples shown by the reference numerals 6B to 6D in FIG. 6, in the head fixation device 100 shown by the reference numerals 5A to 5C in FIG. 5, a large number of through-holes are formed in the temporal region support portion 102. By forming a large number of through-holes, the breathability can be increased, and the comfort of the subject S can be improved. The size and the number of the through-holes are appropriately selected such that the temporal region support portion 102 has strength capable of supporting the temporal region.

In the example shown by the reference numeral 6E in FIG. 6, a large number of through-holes are formed in the temporal region support portion 102 to also serve as the viewing window portion 103. That is, the translucent window formed by a large number of through-holes functions as the viewing window portion 103, and the operator can visually recognize the region 140 from the orbital cavity to the external auditory canal of the temporal region of the subject S through the viewing window portion 103 (see through the viewing window portion 103). In addition, a large number of through-holes also function as ventilation holes, and the comfort of the subject S can also be improved. The size and the number of the through-holes are appropriately selected such that the operator can visually recognize the region 140 and the temporal region support portion 102 has strength capable of supporting the temporal region.

### Modification Example 2 of First Embodiment

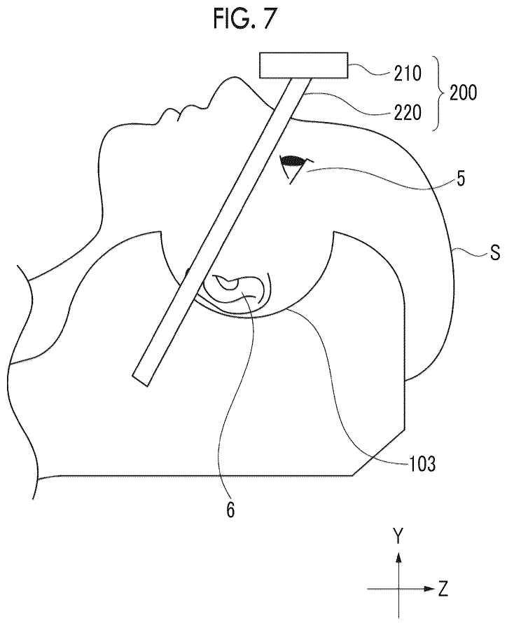

A lens protection member that protects the lens of the eye 5 may be provided in the head fixation device 100 shown in FIGS. 1 to 6. FIG. 7 shows an example of the head fixation device 100 including a lens protection member 200. In FIG. 7, for example, the lens protection member 200 is provided in the head fixation device 100 shown in FIGS. 2 to 4, but the lens protection member 200 may be provided in other head fixation devices 100.

In a case where the subject S is irradiated with the laser from the gantry 2 to perform the registration, the laser enters the eye 5 in a case where the subject S opens the eye 5, and there is a concern that the eye 5 may be injured. In addition, in a case where the medical imaging apparatus 1 is an X-ray CT apparatus, even though the subject S closes the eye 5, the X-ray may adversely affect the lens of the eye 5. Therefore, in order to protect the eye 5 (lens) of the subject S, the lens protection member 200 is provided in the head fixation device 100.

The lens protection member 200 comprises a protective shield portion 210 and an attachment unit 220. The protective shield portion 210 is formed of a material that shields a laser or X-rays so as to protect the eye 5 of the subject S.

The attachment unit 220 is a band-shaped member that is continuously provided in the protective shield portion 210 and that is used to attach the lens protection member 200 to the head fixation device 100. It is desirable that a material having low shielding ability (that is, high transmittance) against X-rays or RF pulses emitted from the medical imaging apparatus 1 is used in the attachment unit 220 so that the influence on the captured tomographic image can be suppressed. Examples of a material of the attachment unit 220 include a resin. Preferably, the attachment unit 220 of the lens protection member 200 has a relatively small width to achieve both the strength capable of holding the protective shield portion 210 and the visibility of the region 140.

In a case where the lens protection member 200 is attached to the head fixation device 100, the protective shield portion 210 is disposed to cover the eye 5 of the subject S. Further, in order to suppress the reduction in the visibility of the region 140 from the orbital cavity to the external auditory canal of the temporal region, it is desirable that the lens protection member 200 is attached to the head fixation device 100 to avoid the region 140 as much as possible.

Preferably, the attachment unit 220 has an attachable and detachable fixing unit (not shown) such as a hook-and-loop fastener (for example, a magic tape (registered trademark)) and/or a button (for example, a snap button). The lens protection member 200 can be attachably and detachably attached to the head fixation device 100 by the fixing unit. For example, the lens protection member 200 can be attached to the head fixation device 100 to protect the eye 5 of the subject S during registration, and the lens protection member 200 can be detached so that the lens protection member 200 does not affect the tomographic image during imaging by the medical imaging apparatus 1.

In addition, in a case where the medical imaging apparatus 1 is an X-ray CT apparatus, the lens protection member 200 may be attached to the head fixation device 100 during imaging in order to protect the eye 5 of the subject S. In this case, it is desirable that the protective shield portion 210 can suppress the influence on the image quality of the tomographic image while protecting the eye 5 (lens) of the subject S from the X-rays emitted from the gantry 2. That is, the protective shield portion 210 is required to have a capability of shielding X-rays from the viewpoint of protecting the eyes 5, but is also required to have a certain degree of X-ray transmittance in consideration of the influence on the image quality. Furthermore, it is desirable that the protective shield portion 210 has a shape that has a sufficient size to cover the eyes 5 and has a width that is not too large in order to reduce the influence on the image quality. In consideration of the request that it is difficult to achieve both of these, the shape and the material of the protective shield portion 210 are appropriately selected so that the X-ray shielding performance is not excessively high.

For example, in consideration of the positions and sizes of both eyes, a rectangular shape having a width of about several centimeters (5 centimeters as a specific example) and a length of about several tens of centimeters (slightly less than 20 centimeters as a specific example) is exemplified as the shape of the protective shield portion 210. A bismuth sheet having a thickness of about several millimeters (2 millimeters as a specific example) may be used as a material of the protective shield portion 210. In addition, an acrylic plate or the like may be used to fix the bismuth sheet. In addition, for example, a position separated from the eye 5 by several centimeters (3 centimeters as a specific example) is exemplified as the position where the protective shield portion 210 is disposed. By setting the shape, the material, and the position of the protective shield portion 210 in this way, it is possible to realize reduction in the influence on the image quality and reduction in the exposure of the eye 5.

### Modification Example 3 of First Embodiment

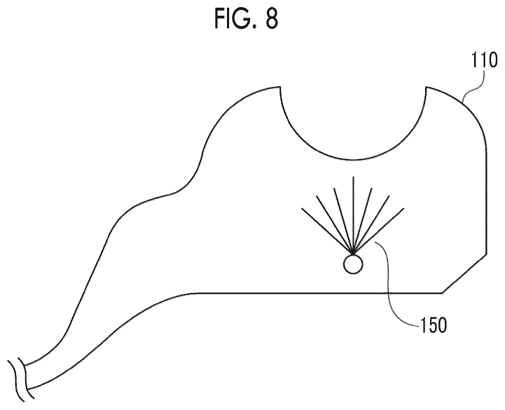

As described above, there are several types of reference lines of the head, and each reference line has a desired inclination angle range with respect to a slice direction in a case of capturing a tomographic image. Therefore, an angle marker that serves as a guide for the inclination angle may be provided in the head fixation device 100. FIG. 8 is a diagram showing an example of the head fixation device 100 including an angle marker 150. In the head fixation device 100 shown in FIG. 8, the angle marker 150 is provided on a surface of the temporal region support portion 102 in the vicinity of the viewing window portion 103.

In a case of registering the head with the predetermined reference line, the operator can check the inclination angle of the gantry 2 with the laser emitted from the gantry 2 and the angle marker 150 while visually recognizing the region 140 from the orbital cavity to the external auditory canal of the temporal region of the subject S through the viewing window portion 103. Therefore, the operator can more easily and accurately perform the registration of the head. In a case of the registration, the operator can understand the horizontal direction by the laser emitted from the gantry 2, but a level indicating the horizontal direction may be further provided on the angle marker 150. Examples of the level include an air bubble tube in which an air bubble is placed in a tube filled with a liquid.

### Second Embodiment

In the first embodiment and Modification Examples 1 to 3, the head fixation device 100 has been described. By the way, in a case of imaging the chest or abdomen of the subject S, an arm fixation device for placing both arms is used in the vicinity of the head of the subject S so that both arms of the subject S do not interfere with the imaging. In addition, depending on the imaging plan, the imaging of the head and the imaging of the chest or the abdomen may be continuously performed. In a case of continuous imaging, the head fixation device 100 described in the first embodiment can also be used in combination with the arm fixation device.

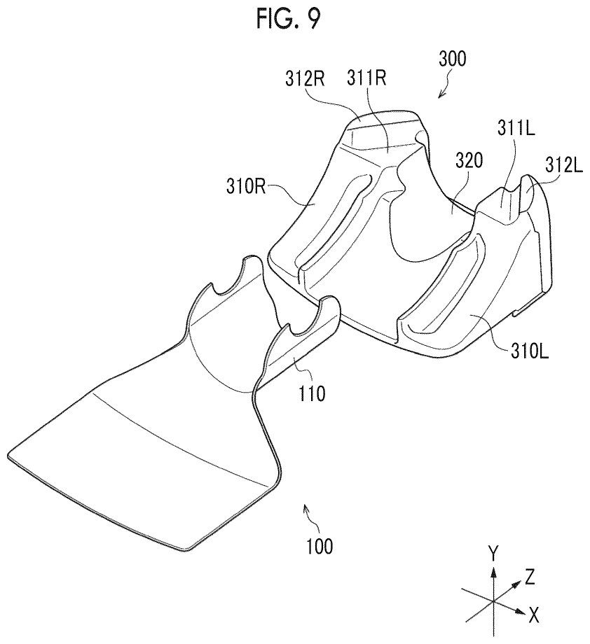

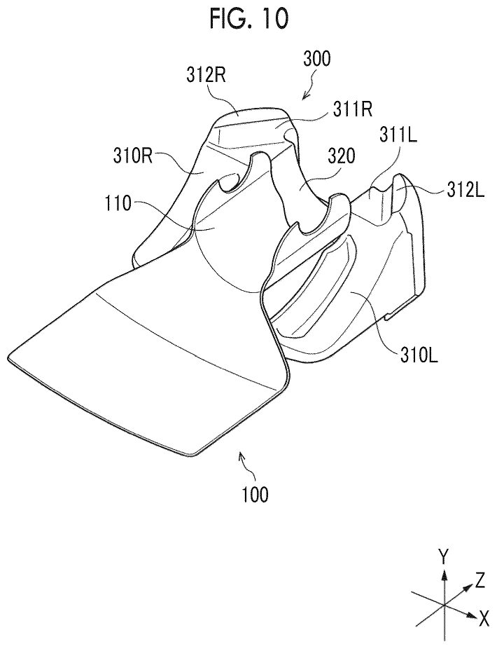

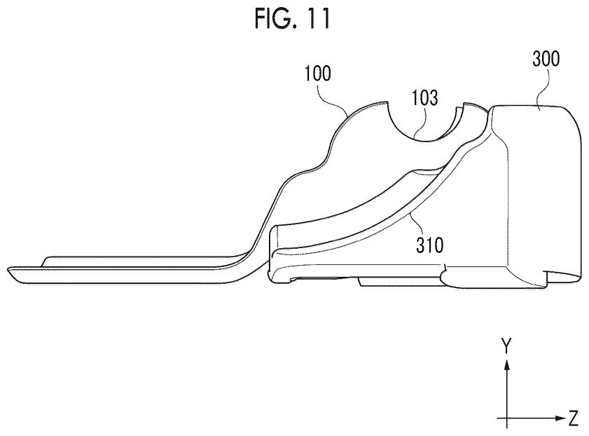

FIGS. 9 to 11 show the head fixation device 100 and an arm fixation device 300. FIG. 9 is an exploded perspective view in a case where the head fixation device 100 and the arm fixation device 300 are used in combination. FIG. 10 is an assembly perspective view showing a state where the head fixation device 100 and the arm fixation device 300 are combined. FIG. 11 is a front view of FIG. 10.

In a case where the imaging of the head and the imaging of the chest or the abdomen are continuously performed, the head fixation device 100 and the arm fixation device 300 are disposed at an end portion (an end portion on the positive side in the Z-axis direction) of the bed 3 on the gantry 2 side in the longitudinal direction in a state where the head fixation device 100 and the arm fixation device 300 are combined. Then, after placing the head of the subject S on the head fixation device 100, the operator performs the registration of the head.

In a case where the continuous imaging is started, in a case of imaging the chest or the abdomen, both arms of the subject S are moved above the head of the subject S and placed on the arm fixation device 300. In a case of imaging the head, both arms of the subject S are moved to both the left and right sides of the body of the subject S. As a result, it is possible to smoothly perform the continuous imaging of the head and the chest or the abdomen.

The material of the arm fixation device 300 is appropriately selected in the same manner as the head fixation device 100 so as to suppress the influence on the image quality of the tomographic image obtained by the medical imaging apparatus 1. Specifically, as the material of the arm fixation device 300, a non-metal having a relatively low absorbance and refractive index with respect to the X-rays or the RF pulses emitted from the medical imaging apparatus 1 is preferable. Examples of the material of the arm fixation device 300 include carbon (carbon fiber).

As shown in FIG. 9, the arm fixation device 300 has a shape similar to a truncated square pyramid as a whole. The arm fixation device 300 includes a pair of upper arm placement portions 310, a pair of forearm placement portions 311, a pair of protruding portions 312, and a receiving portion 320.

The receiving portion 320 is a recess portion formed by hollowing out the vicinity of the center in the X-axis direction of the upper surface of the arm fixation device 300 having a substantially truncated square pyramid shape in the Y-axis direction. Preferably, the receiving portion 320 has a shape corresponding to the head placement portion 110 having a substantially partial circular tube (semicircular tube) shape. The receiving portion 320 has an internal space large enough to accommodate the head placement portion 110 of the head fixation device 100. As shown in FIGS. 10 and 11, in a state where the head fixation device 100 and the arm fixation device 300 are combined, the head placement portion 110 is inserted into the receiving portion 320.

The pair of upper arm placement portions 310 are formed on both sides of the receiving portion 320 in the X-axis direction (both sides of the bed 3 in the lateral direction). The pair of upper arm placement portions 310 are inclined with respect to the Y-axis direction and have a pair of gentle curved surfaces along the roundness of the upper arm of the subject S. One of the pair of upper arm placement portions 310 is a right upper arm placement portion 310R and the other is a left upper arm placement portion 310L.

The pair of forearm placement portions 311 are formed on both sides of the receiving portion 320 in the X-axis direction (both sides of the bed 3 in the lateral direction). The pair of forearm placement portions 311 corresponds to a portion that remains on both sides in the X-axis direction without being hollowed out in the upper surface of the arm fixation device 300 having a substantially truncated square pyramid shape in the Y-axis direction. One of the pair of forearm placement portions 311 is a right forearm placement portion 311R and the other is a left forearm placement portion 311L.

The pair of protruding portions 312 are formed to protrude toward the positive side in the Y-axis direction at both end portions in the X-axis direction that remains without being hollowed out in the upper surface of the arm fixation device 300 having a substantially truncated square pyramid shape in the Y-axis direction. In a case where the subject S placed on the bed 3 in a supine position moves both arms above the head, the upper arm portions of both arms are placed on the upper arm placement portion 310, and the forearm portions of both arms are placed on the forearm placement portion 311. The pair of protruding portions 312 prevents the forearm portions of both arms placed on the forearm placement portion 311 from falling from the forearm placement portion 311. The upper arm portion and/or the forearm portion may be fixed with a band comprising a hook-and-loop fastener as necessary.

As shown in FIG. 11, in a state where the head fixation device 100 and the arm fixation device 300 are combined, the size (the height in the up-down direction) of the upper arm placement portion 310 of the arm fixation device 300 in the Y-axis direction is lower than the size of the viewing window portion 103 of the head fixation device 100. Therefore, the viewing window portion 103 is exposed without being covered by the arm fixation device 300 in front view (in a case of being viewed from the X-axis direction). Therefore, as in the first embodiment and Modification Examples 1 to 3, in the second embodiment as well, the operator can accurately register the head at the predetermined position while visually recognizing the region 140 from the orbital cavity to the external auditory canal of the temporal region of the subject S through the viewing window portion 103.

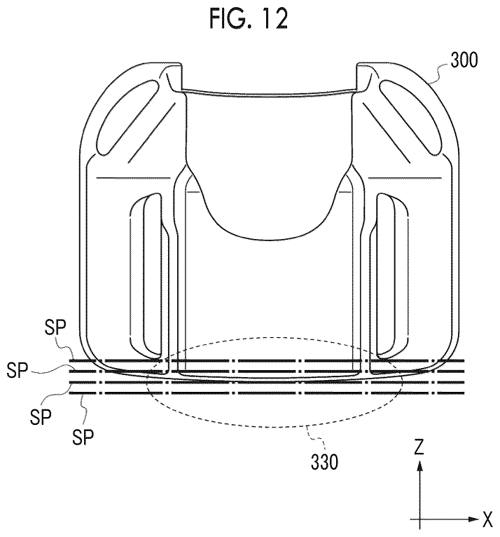

FIG. 12 is a plan view of an arm fixation device 300. In FIG. 12, a portion surrounded by a dotted line corresponds to the end portion 330 of the arm fixation device 300 that is on the negative side in the Z-axis direction (body axis direction). Further, in FIG. 12, a plurality of imaging cross sections (slice planes) SP having different positions in the Z-axis direction are indicated by a one-dot chain line. In general, the medical imaging apparatus 1 acquires a plurality of tomographic images by changing a position in the Z-axis direction. An interval between the plurality of tomographic images in the Z-axis direction is referred to as a slice interval (slice thickness).

As shown in FIGS. 9 to 11, in a case where the head imaging is performed in a state where the head fixation device 100 and the arm fixation device 300 are combined, the end portion 330 is disposed at a position overlapping the head of the subject S in the Y-axis direction. Therefore, the end portion 330 is imaged together with the head of the subject S in a case of capturing the tomographic image.

Here, the end portion 330 of the arm fixation device 300 is not parallel to the imaging cross section (slice plane). As shown in the plan view of FIG. 12, in the XZ plane (in the top view), the contour of the end portion 330 is formed in a curve with respect to the imaging cross section. Preferably, as shown in FIG. 12, the curve forming the contour (outer periphery or outer edge) of the end portion 330 is a curve having a curvature that extends over a plurality of imaging cross sections having different positions in the Z-axis direction. In other words, as shown in FIG. 12, the contour of the end portion 330 is formed in a curve having a length in the Z-axis direction that is several times the slice interval. Preferably, the contour of the end portion 330 is formed in a curve having a length in the Z-axis direction that is three times or more the slice interval.

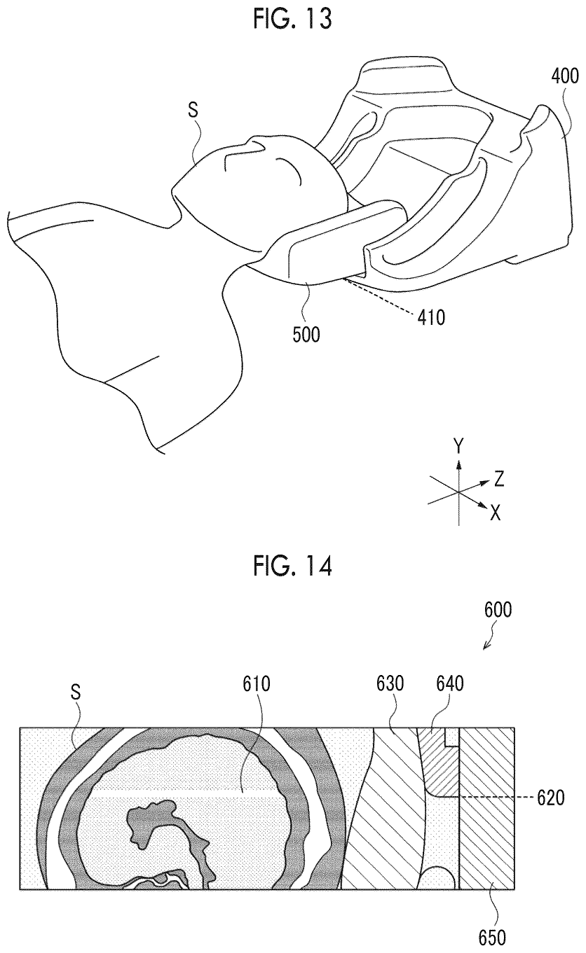

FIG. 13 shows a state where the head of the subject S is placed on a head fixation device 500 in a supine position in a state where the head fixation device 500 and an arm fixation device 400 according to the related art are combined. As shown in FIG. 13, an end portion 410 of the arm fixation device 400 on the negative side in the Z-axis direction is disposed at a position overlapping the head fixation device 500 in the Y-axis direction. Therefore, the end portion 410 is imaged together with the head of the subject S in a case of capturing the tomographic image. Here, although hidden in FIG. 13, in the XZ plane (in the top view), a portion of the contour of the end portion 410 of the arm fixation device 400 that overlaps the head is a straight line of which the contour of the end portion 410 is substantially parallel to the imaging cross section.

FIG. 14 schematically shows a tomographic image 600 of the head of the subject S captured by the medical imaging apparatus 1 in the state shown in FIG. 13. In the tomographic image 600 shown in FIG. 14, a plurality of members that are disposed at a position overlapping the head of the subject S in the Y-axis direction during imaging together with the substantially circular head of the subject S are imaged. Specifically, in the tomographic image 600, a region 630 corresponds to a part of the head fixation device 500, a region 640 corresponds to a part of the arm fixation device 400, and a region 650 corresponds to a part of the bed 3. As shown in the tomographic image 600, a blurred white line appears as an artifact (noise) in the tomographic image of the head of the subject S.

In the tomographic image 600, a portion corresponding to the contour of the end portion 410 of the arm fixation device 400 is indicated by a dotted line 620, and it can be seen that the artifact appears at a position corresponding to the end portion 410 in the Z-axis direction. The inventors have considered that the cause of occurrence of this artifact is an extreme difference in density of a subject (consisting of the subject S and various members) before and after imaging of a linear contour of the end portion 410 of the arm fixation device 400 in a certain imaging cross section. Based on this consideration, in the arm fixation device 300, the contour of the end portion 330 is formed in a curve with respect to the imaging cross section (slice plane). As a result, it is possible to mitigate the extreme difference in density before and after the imaging of the contour of the end portion 410 of the arm fixation device 300. In a case where the curve forming the contour of the end portion 330 extends over a plurality of imaging cross sections having different positions in the Z-axis direction, since the extreme difference in density before and after the imaging of the contour of the end portion 410 of the arm fixation device 300 can be dispersed over the plurality of imaging cross sections, the extreme difference in density can be effectively mitigated.

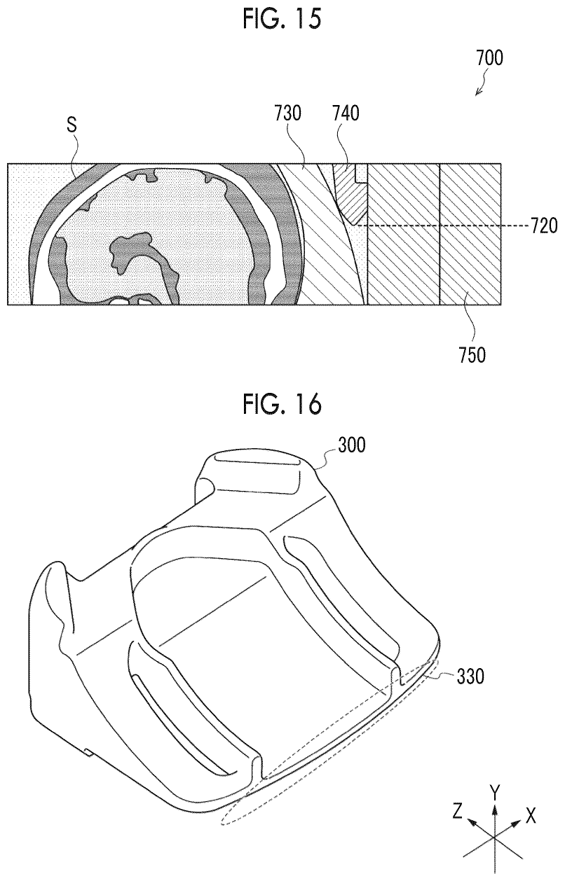

FIG. 15 schematically shows a tomographic image 700 of the head of the subject S captured by the medical imaging apparatus 1 in a state where the head fixation device 100 and the arm fixation device 300 are combined as shown in FIG. 10. In the tomographic image 700 shown in FIG. 15, a plurality of members that are disposed at a position overlapping the head of the subject S in the Y-axis direction during imaging together with the head of the subject S are imaged. Specifically, in the tomographic image 700, a region 730 corresponds to a part of the head fixation device 100, a region 740 corresponds to a part of the arm fixation device 300, and a region 750 corresponds to a part of the bed 3. In the tomographic image 700, a portion corresponding to the contour of the end portion 330 of the arm fixation device 300 is indicated by a dotted line 720. As shown in the tomographic image 700, it can be confirmed that the artifact in the related art that has occurred in the tomographic image of the head of the subject S is suppressed by using the arm fixation device 300.

Therefore, according to the second embodiment, in a case where the imaging of the head and the imaging of the chest or the abdomen are continuously performed, the operator can smoothly perform the imaging after accurately registering the head at a predetermined position while visually recognizing the region 140 from the orbital cavity to the external auditory canal of the temporal region of the subject S through the viewing window portion 103. Further, the image quality of the tomographic image of the head to be obtained can be improved.

### Others

In the second embodiment, a case where the head fixation device 100 and the arm fixation device 300 are used in combination has been described. However, the arm fixation device 300 can also be used alone. FIG. 16 shows another example of the arm fixation device 300. As shown in FIG. 16, in a case where the arm fixation device 300 is used alone, preferably, the arm fixation device 300 may not comprise the receiving portion 320.

Even in a case where the tomographic image of the subject S is captured using the arm fixation device 300 shown in FIG. 16 alone, the end portion 330 of the arm fixation device 300 is disposed at a position overlapping the subject S in the Y-axis direction. Since the contour of the end portion 330 of the arm fixation device 300 is formed in a curve with respect to the imaging cross section, even in a case where the end portion 330 of the arm fixation device 300 is included in the imaging range, it is possible to suppress the artifact generated by the contour of the end portion 330.

It is needless to say that the present invention is not limited to the above-described embodiments and can be variously modified. For example, in the second embodiment, a case where the head fixation device 100 and the arm fixation device 300 are configured as separate bodies has been described, but the head fixation device 100 and the arm fixation device 300 may be integrally formed.

### EXPLANATION OF REFERENCES

100: head fixation device
101: occipital region placement portion
102, 102L, 102R: temporal region support portion
103, 103L, 103R: viewing window portion
110: head placement portion
140: region
150: angle marker
200: lens protection member
210: protective shield portion
300: arm fixation device
310: upper arm placement portion

What is claimed is:

1. A head fixation device for use in a medical imaging apparatus, comprising:
an occipital region placement portion on which an occipital region of a head of a subject is placed; and
a temporal region support portion that is continuously provided from both sides of the occipital region placement portion and supports both sides of a temporal region of the head,
wherein the temporal region support portion includes a viewing window portion which is configured so that a region of the temporal region, which is a reference in a case of capturing a tomographic image of the head, is visually recognizable in a state where the occipital region is placed on the occipital region placement portion.

2. The head fixation device according to claim 1,
wherein the region of the temporal region that is visually recognizable is a region from an orbital cavity to an external auditory canal.

3. The head fixation device according to claim 1,
wherein the reference is any one of an orbitomeatal base line, a Reid's base line, an anterior commissure-posterior commissure line, a superior orbitomeatal line, or an auricular line.

4. The head fixation device according to claim 2,
wherein the reference is any one of an orbitomeatal base line, a Reid's base line, an anterior commissure-posterior commissure line, a superior orbitomeatal line, or an auricular line.

5. The head fixation device according to claim 1,
wherein the viewing window portion is an opening or a cutout provided in the temporal region support portion.

6. The head fixation device according to claim 2,
wherein the viewing window portion is an opening or a cutout provided in the temporal region support portion.

7. The head fixation device according to claim 5,
wherein the opening or the cutout is formed in a curve.

8. The head fixation device according to claim 6,
wherein the opening or the cutout is formed in a curve.

9. The head fixation device according to claim 1,
wherein the temporal region support portion has breathability.

10. The head fixation device according to claim 2,
wherein the temporal region support portion has breathability.

11. The head fixation device according to claim 9,
wherein the breathability of the temporal region support portion is achieved by a plurality of ventilation holes.

12. The head fixation device according to claim 10,
wherein the breathability of the temporal region support portion is achieved by a plurality of ventilation holes.

13. The head fixation device according to claim 1,
wherein the viewing window portion has a net shape provided on the temporal region support portion.

14. The head fixation device according to claim 1, further comprising:
a lens protection member that protects eyes of the subject.

15. The head fixation device according to claim 14,
wherein the lens protection member is attachably and detachably disposed on the temporal region support portion.

16. The head fixation device according to claim 1, further comprising:
an upper arm placement portion on which an upper arm portion of the subject is placed.

17. The head fixation device according to claim 16,
wherein the upper arm placement portion is a separate body from the occipital region placement portion and the temporal region support portion.

18. The head fixation device according to claim 17,
wherein, in a case where the head fixation device is used, at least a part of the upper arm placement portion is disposed below the occipital region placement portion.

19. The head fixation device according to claim 18,
wherein, in a case where the head fixation device is used, among a plurality of end portions of the upper arm placement portion, an end portion located below the occipital region placement portion is curved.

20. The head fixation device according to claim 19,
wherein the curved end portion of the upper arm placement portion located below the occipital region placement portion extends over three or more tomographic images.
